(19) United States
(12) Patent Application Publication
(10) Pub. No.: US 2013/0327338 A1
(43) Pub. Date: Dec. 12, 2013

(54) INTRA-UTERINE DEVICE AND RELATED METHODS

(71) Applicant: HOLOGIC, INC., Marlborough, MA (US)

(72) Inventors: William L. Churchill, Bolton, MA (US); James Duronio, Westford, MA (US); Matthew LaPlaca, Cumberland, RI (US); Mark Putnam, Weymouth, MA (US)

(21) Appl. No.: 13/914,281

(22) Filed: Jun. 10, 2013

Related U.S. Application Data
(60) Provisional application No. 61/658,000, filed on Jun. 11, 2012.

(51) Int. Cl. A61F 6/14

(52) U.S. Cl. CPC A61F 6/144
USPC 128/833

(57) ABSTRACT

An intrauterine device includes an elongate member; and a flexible arm having a first end and a second end, wherein the first end is operatively coupled to the elongate member at a first location, and wherein the second end is operatively coupled to the elongate member at a second location, and wherein the flexible arm is adjustable between a closed configuration, in which the flexible arm is closely apposed to the elongate member, and an open configuration, in which the flexible arm extends away from the elongate member, and wherein the flexible arm is biased to be in the closed configuration.

### RELATED APPLICATION DATA

The present application claims the benefit under 35 U.S.C. §119 to U.S. Provisional Patent Application Ser. No. 61/658,000, filed on Jun. 11, 2012. The foregoing application is hereby incorporated by reference into the present application in its entirety.

### FIELD OF THE INVENTION

The technology disclosed herein generally relates to medical implants and, more specifically, to intrauterine devices and related delivery devices and systems, as well as methods of using the same.

### BACKGROUND

According to 2010 census data, there are over 60 million women of reproductive age in the United States. Seven in ten women in this age bracket are sexually active and wish to avoid pregnancy. See Guttmacher Institute, Facts on Contraceptive Use in the United States, June, 2010 (available at http://www.guttmacher.org/pubs/fb contr use.html, accessed Apr. 10, 2012). Accordingly, a large majority of women of reproductive age in the U.S. have used at least one form of temporary birth control.

A growing number of women use intrauterine devices (IUDs) for temporary birth control. IUDs typically include a flexible body, often T- U- or V shaped and roughly 1½ inches long, and a contraceptive agent such as copper or progesterone. When deployed within a patient's uterus, IUDs work by inhibiting sperm motility (copper) or by providing depot release of contraceptive steroid (progesterone); they may also irritate the uterine wall, inhibiting embryo implantation. IUDs provide highly effective birth control over long periods of time, and require limited patient care. The potential exists, however, for uterine trauma and patient discomfort during the insertion of IUDs. In addition, currently available “normally open” IUDs require a relatively large-diameter cervical access for delivery, which may increase discomfort for nulliparous patients and for patients with narrow cervixes. There is a need in the art for systems and methods that decrease the potential for discomfort and trauma during IUD insertion.

### SUMMARY OF THE INVENTION

Embodiments of the current invention help to address the needs described above by providing IUDs and IUD delivery systems, and methods of using the same.

In one embodiment, the present invention comprises an intrauterine device. The device comprises an elongate member and a flexible arm having a first end and a second end. The first end of the arm is operatively coupled to the elongate member at a first location, and the second end of the arm is operatively coupled to the elongate member at a second location. In certain embodiments, the flexible arm is adjustable between a closed configuration in which the flexible arm is closely apposed to the elongate member and an open configuration in which the flexible arm extends away from the elongate member. In certain embodiments, the flexible arm is adjustable between a closed configuration in which the first location and the second location define a closed length and an open configuration in which the first location and the second location define an open length, wherein the open length is less than the closed length, and wherein the flexible arm is biased to be in the closed configuration.

In yet another aspect, the present invention comprises methods of preventing conception in a female patient by the delivery of the intrauterine devices according to embodiments of the present invention.

### DESCRIPTION OF THE DRAWINGS

FIGS. 1A-C are schematic and cross-sectional depictions of an intrauterine device according to certain embodiments of the invention.

FIGS. 2A-B are schematic depictions of intrauterine devices in respective closed and open configurations according to embodiments of the invention.

FIG. 3A-B are schematic and cross-sectional depictions, respectively, of an intrauterine device in a closed configuration according to an embodiment of the invention.

FIG. 4 is a schematic depiction of an intrauterine device during deployment, according to certain embodiments of the invention.

FIG. 5 is a schematic depiction of an intrauterine device after deployment and in an open configuration, according to certain embodiments of the invention.

FIG. 6 is a schematic cross-sectional depiction of an intrauterine device and a delivery device according to certain embodiments of the invention.

FIG. 7 is a schematic depiction of an intrauterine device in the open, deployed configuration according to certain embodiments of the invention

FIG. 8 is a schematic depiction of an intrauterine device and a delivery device according to certain embodiments of the invention

FIG. 9 is a schematic depiction of an intrauterine device in a closed configuration according to certain embodiments of the invention.

FIG. 10 is a schematic depiction of an intrauterine device including retention features according to certain embodiments of the invention.

FIG. 11 is a schematic depiction of an intrauterine device with integral thread cutting according to certain embodiments of the invention.

FIG. 12 is a schematic depiction of an intrauterine device including steerable features according to certain embodiments of the invention.

FIG. 13 is a schematic depiction of an intrauterine device including steerable features according to certain embodiments of the invention.

FIG. 14 is a schematic depiction of an intrauterine device including collapsible features according to certain embodiments of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Various embodiments of the disclosed inventions are described hereinafter with reference to the figures. The figures are not necessarily drawn to scale, the relative scale of select elements may have been exaggerated for clarity, and elements of similar structures or functions are represented by like reference numerals throughout the figures. It should also be understood that the figures are only intended to facilitate the description of the embodiments, and are not intended as an exhaustive description of the invention or as a limitation on the scope of the invention, which is defined only by the appended claims and their equivalents. In addition, an illustrated embodiment of the disclosed inventions needs not have all the aspects or advantages shown. An aspect or an advantage described in conjunction with a particular embodiment of the disclosed inventions is not necessarily limited to that embodiment and can be practiced in any other embodiments even if not so illustrated.

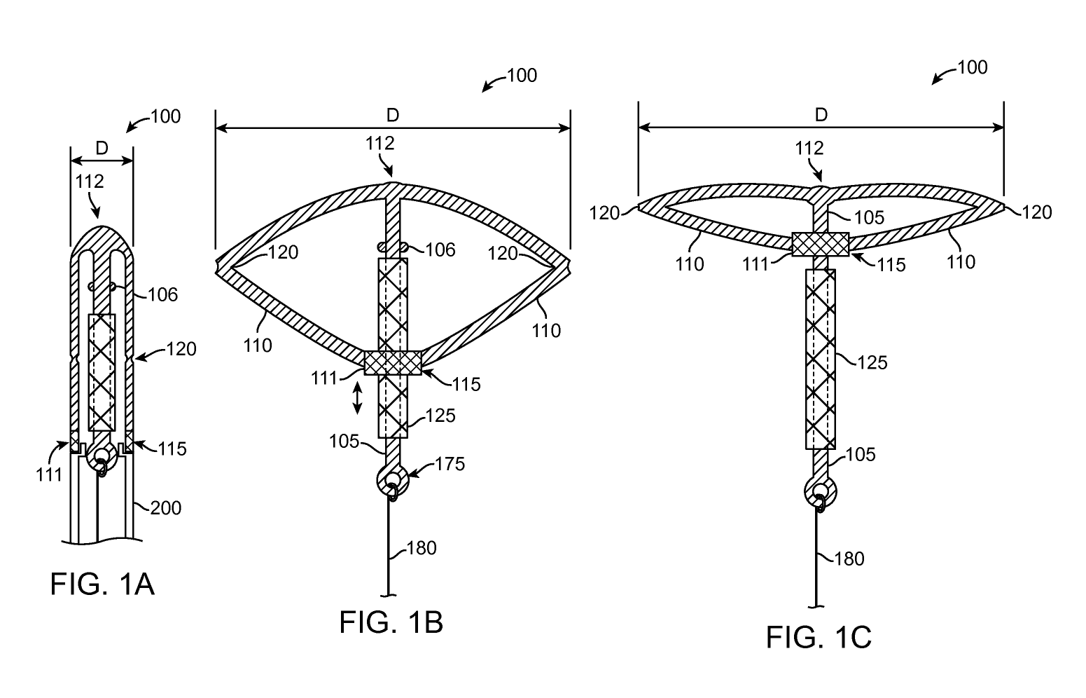

As shown in FIGS. 1A-C, in an exemplary embodiment, intrauterine device 100 includes an elongate member 105 and at least one, and preferably two or more, flexible arms 110 characterized by first 111 (i.e. proximal) and second 112 (i.e. distal) ends. The elongate member 105 may be solid or hollow; may be rigid, semi-rigid, or flexible; and is made from any suitable material. In some embodiments, the elongate member 105 is a thread. Member 105 is described herein as “elongate” as it is generally characterized as having a length longer than its width. Although in a preferred embodiment the elongate member 105 is cylindrical and includes a round cross-sectional shape, in other embodiments it is characterized by a different cross-sectional shape, such as triangular, square, rectangular, or other suitable polygonal shape. The arms 110 are preferably made of any suitable polymeric material. The arms 110 and/or the elongate member 105 may comprise biocompatible polymeric, metallic or alloys material, or combination thereof. The arms 110 and/or the elongate member 105 preferably comprise, carry and/or are coated with copper, agents or other materials (active agent 125) that inhibit sperm mobility or otherwise impart contraceptive properties.

The first and second ends 111, 112 of the at least one flexible arm 110 are operatively coupled to the elongate member 105. As used herein, “operatively coupled” means that the ends 111, 112 are fixed to the elongate member 105 or are slide-able or otherwise moveable with respect thereto. For example, in certain embodiments, the first end 111 is fixed to the elongate member 105 and the second end 112 is shaped as a ring or similar suitable shape that slides over the elongate member 105 comprising or carrying active agent 125. In other embodiments, the second end 112 is fixed to the elongate member 105 and the first end 111 is shaped as a ring or similar suitable shape that slides over the elongate member 105 comprising or carrying active agent 125. In yet other embodiments, neither of the ends 111 and 112 are fixed to the elongate member 105 or to the active agent 125. Instead both ends 111 and 112 are slideable or otherwise moveable with respect thereto. In all embodiments, however, at least one of the first and second ends 111, 112 are moveable with respect to the other end so that the length between them is shortened and the intrauterine device 100 is moved from a closed to an open configuration for use in the body of a patient, as further described herein.

The intrauterine device 100 is moveable between at least two configurations: open (i.e. deployed) and closed (i.e. delivery). In the closed configuration as shown, for example, in

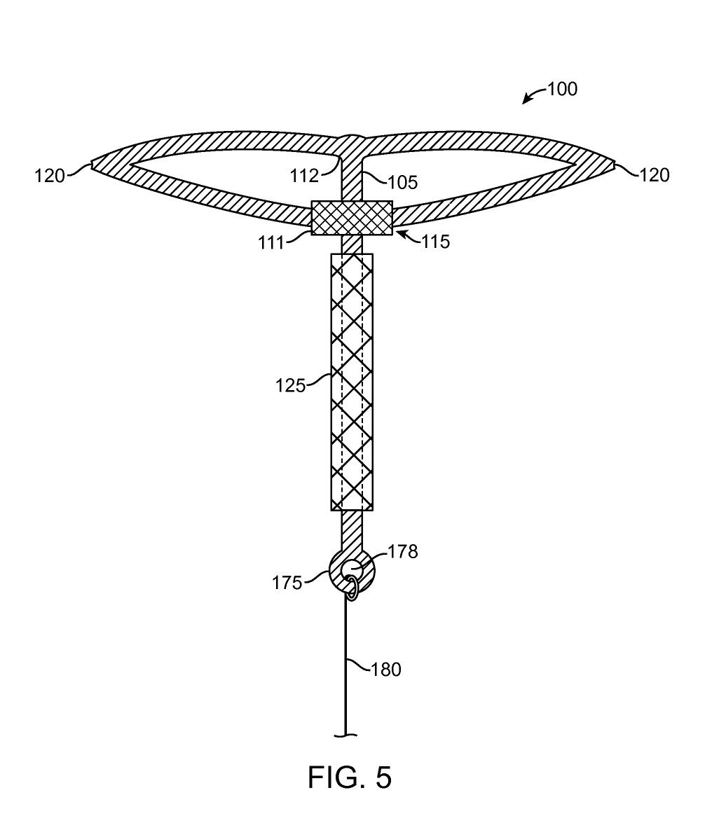

FIGS. 1A, 2A, 3A and 6 the first and second ends 111, 112 are separated by a maximum distance, and the flexible arms 110 are substantially collapsed such that they are closely apposed with the elongate member 105 between the first and second ends 111, 112. As used herein, “closely apposed” means that the flexible arms 110 are substantially proximate to, and/or in direct contact with, the elongate member 105 and/or active agent 125, along substantially the entire length of the flexible arms 110. In the open configuration, as shown for example in FIGS. 1C and 5, the first and second ends 111, 112 are closer together, and the flexible arms 110 extend away from the elongate member 105 and active agent 125. In a preferred embodiment, the intrauterine device 100 is molded or otherwise manufactured in its closed configuration, is subsequently deformed into its open configuration during opening in accordance with the deployment procedure of the present invention, and is held in this open configuration during its useful lifetime within a patient. Manufacturing the device 100 in its closed configuration allows for a smaller diameter device, and therefore easier and more comfortable insertion, when compared to conventional intrauterine devices.

In certain embodiments, the flexible arms 110 are connected to one another at the first end 111 to form a ring or sliding collar 115 and, at the second end 112 by suitable means discussed in further detail below. In an alternate embodiment, the sliding collar 115 abuts the flexible arms 110 at the first end 111, but is a separate structural element that “pushes” the flexible arms as collar 115 slides distally along the elongate member 105 comprising or carrying the active agent 125 and then locks the arms into an open configuration as used in a patient.

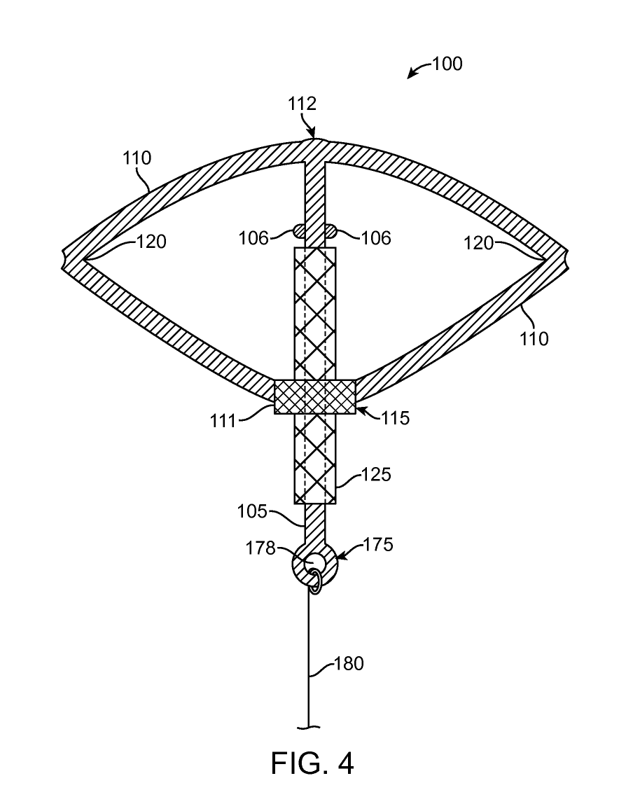

Moreover, the sliding collar 115 may be either slidably coupled with the elongate member 105, as is depicted in FIGS. 1B and 4, or the sliding collar 115 and the elongate member 105 may be fixedly attached to one another as further described herein.

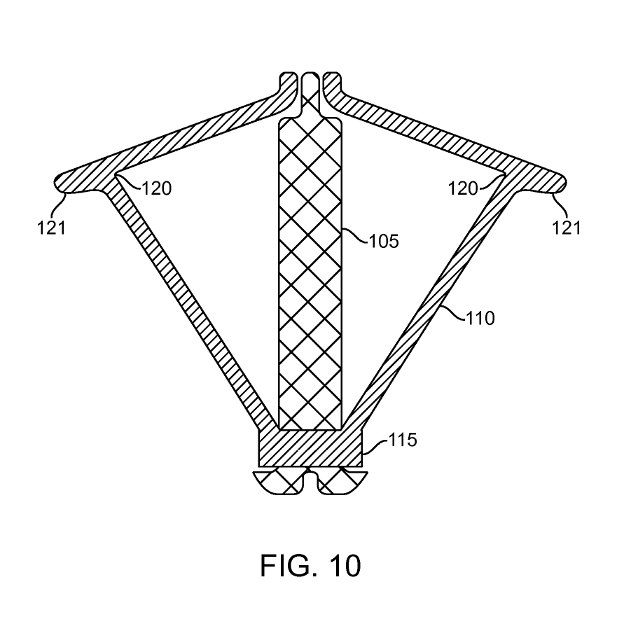

Each flexible arm 110 may optionally include one or more hinged regions 120 which permit the flexible arms 110 to hinge, or otherwise assist the arms to bend, when the first and second ends 111, 112 are brought together and the intrauterine device 100 moves into an open position. The hinged regions 120 may be formed by any suitable means. For example, the hinged regions 120 may be regions of the flexible arms 110 with a reduced diameter relative to the non-hinging portions of the flexible arms 110, or the hinged regions 120 may comprise scoring, perforations, springs, or a material with a lower Young's modulus than the remainder of the flexible arms 110. In other embodiments, the hinged regions 120 are heat treated to allow the arms 110 to flex in these areas. In preferred embodiments, the hinged regions 120 deploy to form rounded “elbows” or deflection areas rather than sharp points. In some embodiments, such as the one shown in FIG. 10, the flexible arms include one or more retention features 121 disposed at the hinged regions 120. Such retention features may extend outwardly as shown in FIG. 10, and may be angled in any suitable direction to help facilitate the retention of the device 100 in a preferred location within the uterus. In certain embodiments, the retention features 121 comprise materials or surface features such as raised portions that are configured to grip or otherwise adhere to tissue. While the present invention is specifically described herein as having two flexible arms, it should be appreciated that the invention may include three, four, or more arms, or in other embodiments, only a single arm that extends partially or completely around the elongate member 105 in an umbrella-like configuration.

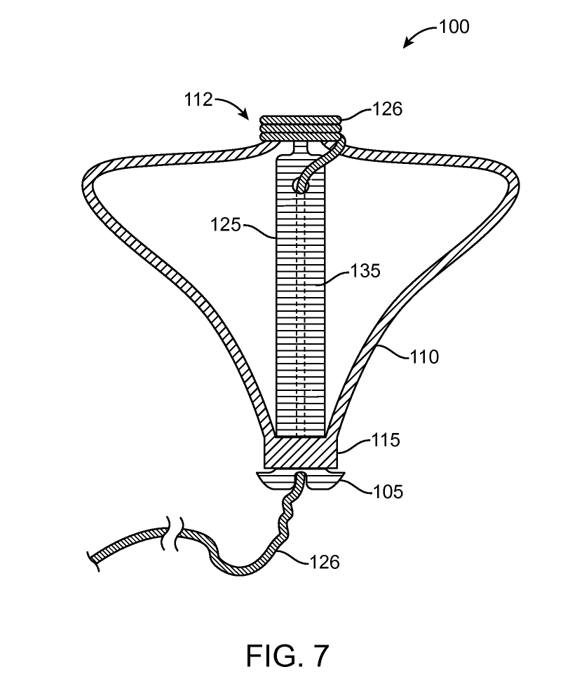

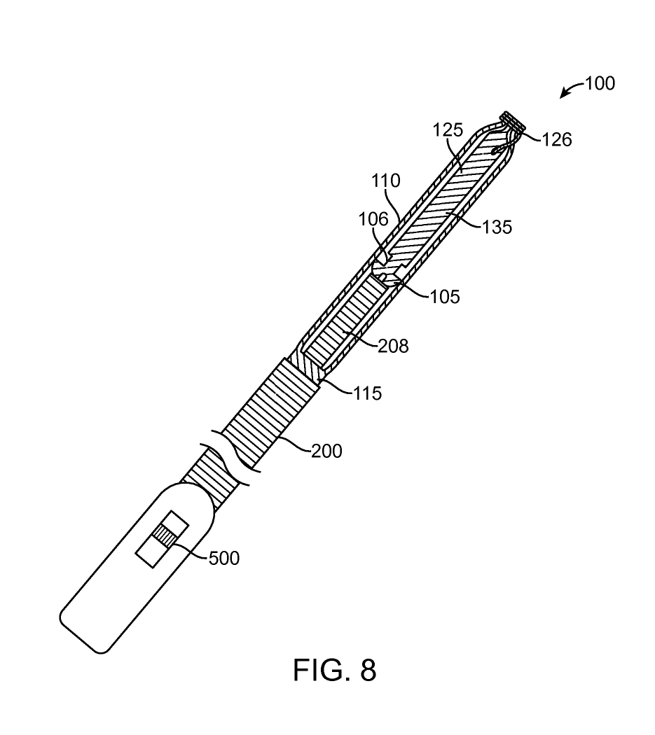

In certain embodiments, the flexible arms 110 are connected at the second end 112 to a structure that extends through a hollow elongate member 105, such as a rod 135 (as shown throughout the figures, such as FIGS. 7-9) or one or more threads 126 (as shown in FIG. 7). In various embodiments, the flexible arms 110 are integrally connected to the rod 135, as shown in FIGS. 7-8. The thread 126 extends through the elongate member 105 and/or rod 135 and can be attached to a portion of the elongate member 105 and/or rod 135, as shown in FIGS. 7-9. As used herein, “thread” is used synonymously with “tether,” “suture,” “pull strings,” or other similar structure that can be manipulated by a user. The elongate member 105 and/or rod 135 may also include a suture anchor 175 having a hole 178 for securing the IUD 100 to a suture 180 for the removal of the IUD from the patient (as shown in FIGS. 1A-C, 3A, 4 and 5).

In the embodiments depicted in FIGS. 7-9, the flexible arms 110 are secured to one another at the second end 112 by means of one or more threads 126, which may extend through the elongate member 105 and/or rod 135. In some embodiments, the threads 126 may be releasably secured to the second end 112 of the flexible arms 110, so that they can be released from the second end 112 if the user applies tension to the threads 126 from the proximal end 111 of the device 100. The reversible attachment of the threads 126 to the second end 112 of the flexible arms 110 may be achieved by any suitable means, for example, the pull string 126 may be looped repeatedly around the second end 112, and/or each of the flexible arms 110 may include a breakable tab positioned at the second end 112 so as to secure the flexible arms 110 to the threads 126 or to one-another. Upon the release of the threads 126 from the flexible arms 110, the flexible arms will elongate and lay in close apposition to the elongate member 105, as is discussed in more detail below.

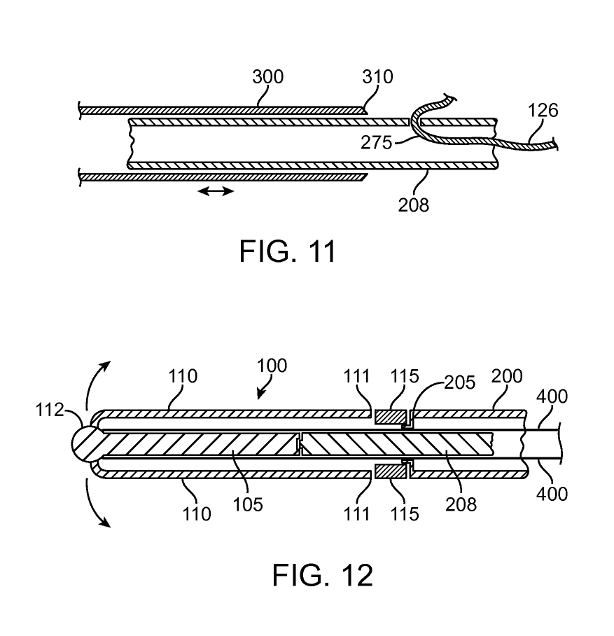

In any embodiment of the present invention that employs one or more threads 126, the delivery device 200 may include means for cutting the threads 126. In one embodiment as shown in FIG. 11, the delivery device 200 includes a cylindrical cutter or hypotube 300 having a distal end with a cutting edge 310 that may be advanced over the delivery device 200 such that it cuts the thread 126. In such embodiments, the delivery device 200 preferably includes a side hole 275 through which the thread 126 is placed such that it is in the path of the hypotube 300 as is advanced over the delivery device 200. In a preferred embodiment, the cutting edge 310 is designed such that the thread 126 curls at the cut end, thus creating a blunt tip at any portion of the thread 126 remaining in the patient's body. In some embodiments, the delivery device 200 includes a spring-loaded mechanism for the rapid and repeatable advance of hypotube 300.

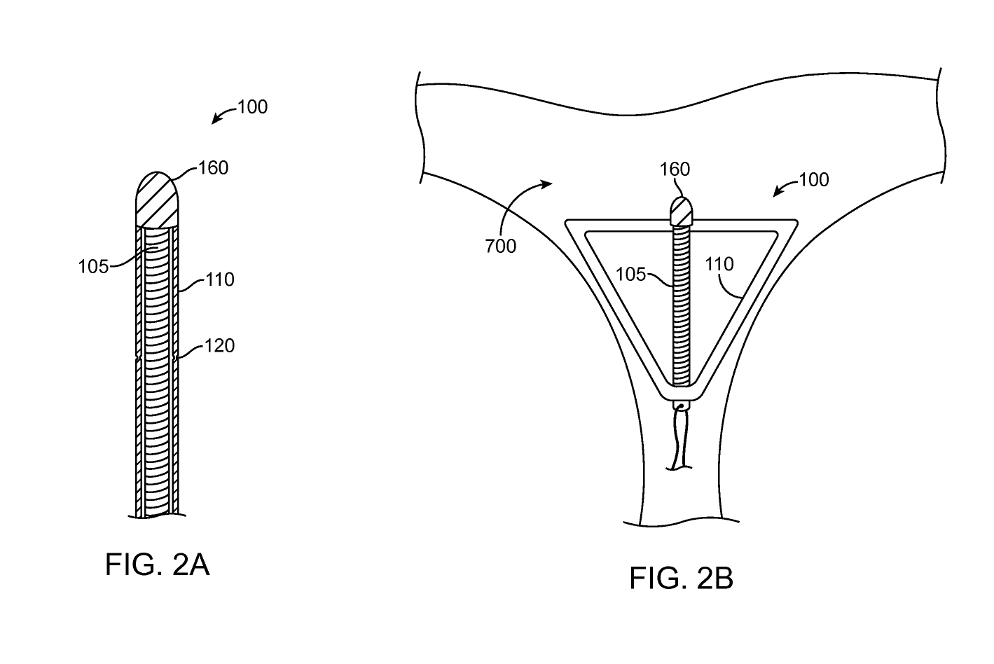

The elongate member 105 optionally includes one or more stop members 106, as shown in FIGS. 1A-B, 3A, 4, and 8, to form a mechanical interaction with collar 115 (if used) or the first or second ends 111, 112 of the one or more flexible arms 110, to lock the intrauterine device 100 in the open position and prevent its collapse into the closed position. The stop members 106 can be formed as regions of increased diameter, or can be formed as balls, protrusions or other atraumatic shapes (as shown in FIGS. 1A-B, 3A, 4). Alternatively, the stop members 106 can be formed as region of decreased diameter, such as a recess, grove, or cuts (as shown in FIG. 8). In embodiments where the elongated member 105 includes thread 126, the stop member 106 is preferably a knot in the thread 126. The IUD 100 also optionally includes an atraumatic tip 160 to prevent trauma to the uterus 700 during insertion as shown in FIGS. 2A-B, and the atraumatic tip 160 may function as a stop member in some embodiments.

The intrauterine device 100 preferably includes at least one contraceptive active agent 125, which is preferably disposed on a sidewall of the elongate member 105 so that, when the intrauterine device 100 is in the closed configuration, the flexible arms 110 cover, or at least partially cover, the contraceptive agent 125. Any suitable contraceptive agent may be used, such as copper, which may be applied to the elongate member 105 and/or to the flexible arms 110 by any suitable means, such as a wrapping of copper wire, copper plating, copper coating, a copper sleeve, etc. Alternatively, the contraceptive agent 125 may be progesterone, which may be incorporated into a progesterone-eluting portion of the sidewall of the elongate member 105 or as a coating thereon.

The intrauterine device also preferably includes at least one removal string 180, which may be attached to the structure extending through the base member, e.g. the rod 135, or it may be one or more of the threads 126. In some embodiments, the rod 135 includes a ring 175 or eyelet 178 through which a pull string 180 can be inserted and/or tied or otherwise secured (as shown in FIGS. 1A-C, 3A, 4 and 5).

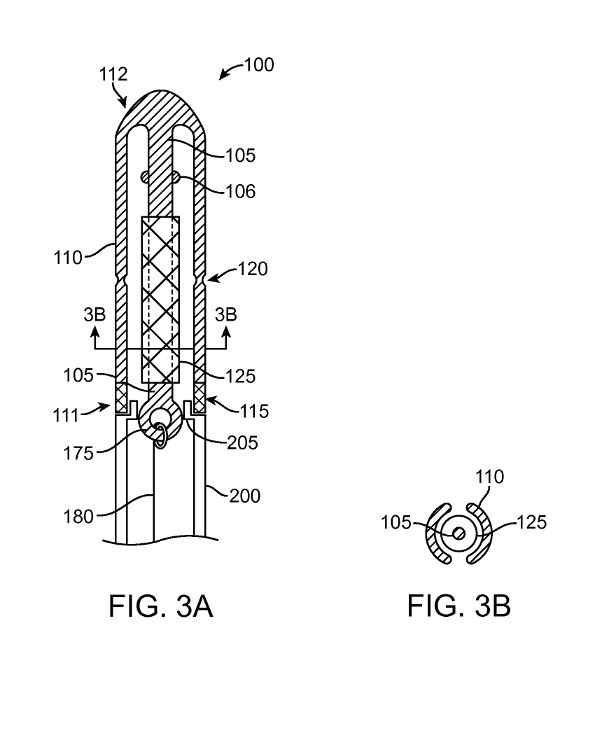

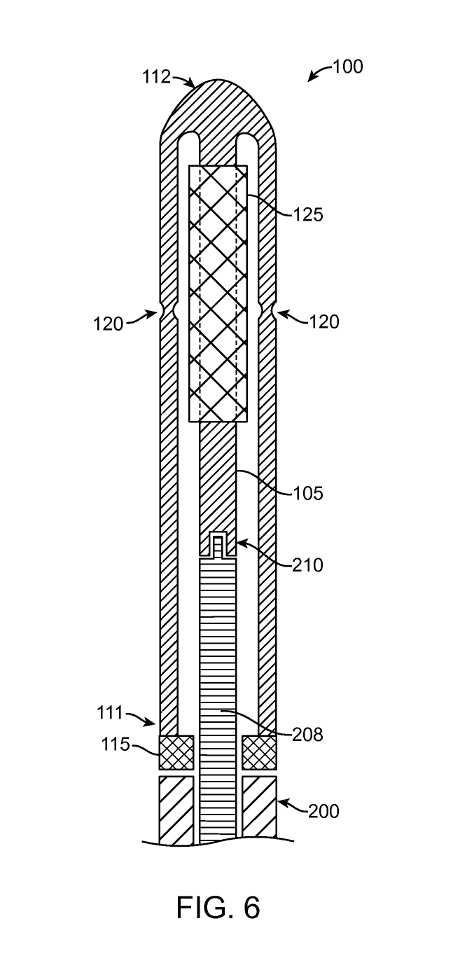

In use, the intrauterine device 100 is delivered to the uterus of a patient with the aid of a delivery device 200. Prior to delivery, an intrauterine device 100 in the closed configuration is coupled to the delivery device 200, as at least partially shown in FIGS. 1A, 3, 6, 8 and 12, so that it can be inserted through the lower genital tract, and the cervix. In one embodiment, the delivery device 200 includes an inner member 208 that forms a mechanical fit 210 with the elongate member 105 of the intrauterine device 100, as shown in FIGS. 6 and 12. In the closed configuration, the flexible arms 110 of the intrauterine device 100 are closely appositioned to the elongate member 105 and active agent 125, and the first and second ends 111, 112 are separated such that the maximal cross-sectional dimension D (such as a diameter) of the intrauterine device 100 is minimized and IUD 100 is easily insertable through a cervix. The sliding collar 115 (if used) or the first end 111 of the at least one flexible arm 110 of the intrauterine device 100 engages the delivery device 200 at its tip 205 (as shown in FIG. 3), which tip is sized and shaped to engage the sliding collar 115. The delivery device 200 is also shaped to accommodate, and optionally to securely engage, the elongate member 105 or an end of a structure extending through the elongate member 105, for instance the rod 135 or the one or more threads 126. The delivery device 200 maintains contact with the device 100 by any suitable means such as, for example, a mechanical lock, interference fit, ball and socket arrangement, threaded interface, or other suitable means. In some embodiments, the tip 205 of the delivery device 200 engages the elongate member 105.

After the intrauterine device 100 is positioned within the uterus where it will be deployed, the first and second ends 111, 112 are brought towards each other to move the intrauterine device 100 into the open (“deployed”) configuration, shown in FIGS. 1C and 5, so that the flexible arms 110 extend away from the elongate member 105 so as to increase the maximal cross-sectional dimension D of the IUD 100, thereby exposing the contraceptive agent (when used) on the sidewall of the elongate member 105. This may be done by actuating an adjustment means of the delivery device 200, which, in turn, adjusts the length between the first and second ends 111, 112 by pushing the first end 111 of the arm(s) 110 towards the second end 112, and/or by pulling the second end 112 of the arm(s) 110 towards the first end 111, or by pushing the sliding collar 115 (if used) toward the second end 112 of the intrauterine device 100, and/or by pulling the structure extending through the elongate member 105 (e.g. the rod 135 or the pull string(s) 126) so that the second end 112 moves toward the sliding collar 115. In an alternate embodiment, a spring extends between first and second ends 111, 112, which spring is in an extended configuration when the device 100 is in a closed configuration and is released to a biased configuration to pull the first and second ends 111, 112 towards each other and urge the device 100 towards its open configuration. During deployment, either of the first or second ends 111, 112, or the sliding collar 115 is optionally moved over a stop member(s) 106, locking the intrauterine device 100 in the open configuration. In some embodiments, where the intrauterine device includes a rod 125, the rod optionally includes one or more stop members 106 in the form of ridges or protrusions that engage with the first and/or second ends 111, 112, or the sliding collar 115, to lock the intrauterine device in the open position.

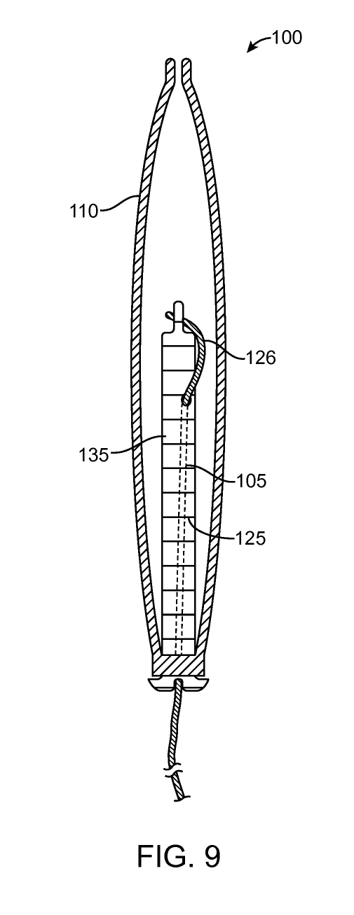

In intrauterine devices 100 that include one or more threads 126 connecting the flexible arms 110 at the second end 112, the device may be collapsed to facilitate removal by releasing the threads 126 from the second end 112, allowing the flexible arms 110 to collapse into a closed position as shown in FIG. 9. In other embodiments, the device may be removed from the uterus 700 by pulling on the rod 135 or elongate member 105 with threads 126 or other suitable means, as shown in FIG. 14. In embodiments in which the device 100 is not directly attached to a sliding collar 115 as previously described, the proximal end 111 of the device 100 is able to pull away from the sliding collar 115 during removal, thus allowing the arms 110 to collapse and fold in a distal direction, as shown in FIG. 14.

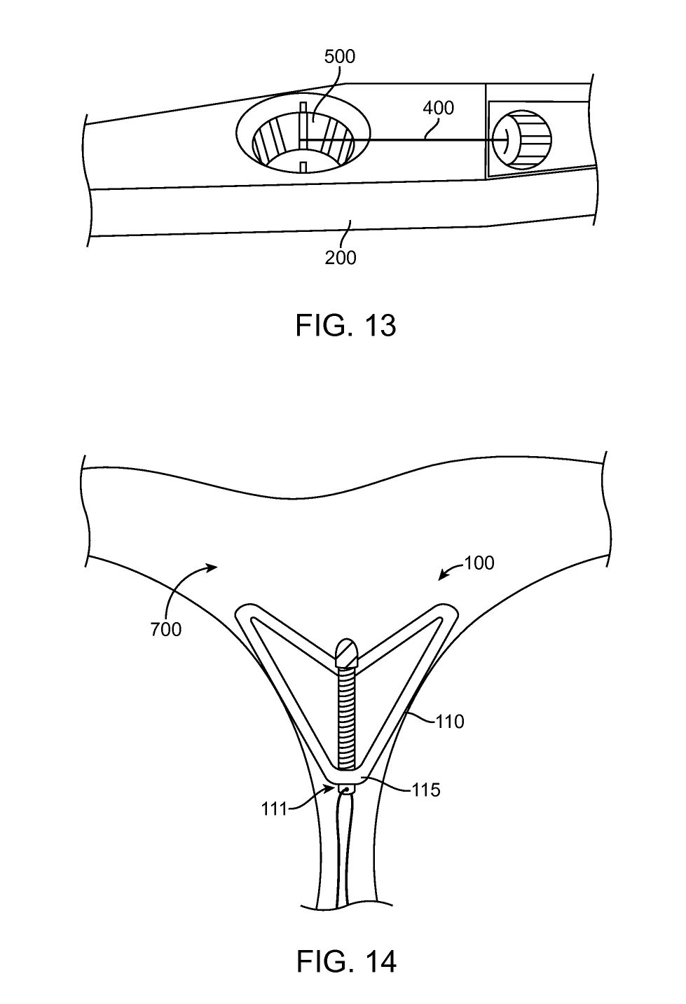

The delivery devices of the present invention are preferably steerable, such as by the intended flexure of the tip 205 of the delivery device 200 or the second end 112 of the intrauterine device 100, as shown in FIG. 12. The ability to steer or bend the delivery device 200 and/or the intrauterine device 100 is achieved by any suitable means such as, for example, the use of one or more pull strings 400, as shown in FIG. 12. Such pull strings 400 are attached to the delivery device 200 and/or the intrauterine device 100 by any suitable means, such as by using an adhesive material, by mechanical lock, or by winding the pull string(s) around a portion of the delivery device 200 and/or the intrauterine device 100. By selectively applying tension to one or more of the pull strings 400, the tip of the delivery device 200 and/or the intrauterine device 100 is deflected in a desired direction, as shown by the arrows in FIG. 12. In one embodiment, the one or more pull strings 400 interact with a rotatable element 500 mounted within the delivery device 200, as shown in FIG. 13. The rotatable element 500 preferably includes a groove or other means to facilitate the gripping and movement of the one or more pull strings 400 by a user.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the scope of the invention. Accordingly, the described embodiments are to be considered in all respects as only illustrative and not restrictive.

What is claimed is:

1. An intrauterine device, comprising:
an elongate member; and
a flexible arm having a first end and a second end,
wherein the first end is operatively coupled to the elongate member at a first location, and the second end is operatively coupled to the elongate member at a second location,
wherein the flexible arm is adjustable between a closed configuration, in which the flexible arm is closely apposed to the elongate member, and an open configuration, in which the flexible arm extends away from the elongate member, and
wherein the flexible arm is biased to be in the closed configuration.

2. The intrauterine device of claim 1, wherein the first location and the second location define a length, and wherein the length when the flexible arm is in the closed configuration is greater than the length when the flexible arm is in the open configuration.

3. The intrauterine device of claim 2, further comprising adjustment means for adjusting said length.

4. The intrauterine device of claim 2, further comprising:
a collar slideably coupled to the elongate member and disposed proximal to the first location; and
a stop member axially fixed to the elongate member and disposed distal to the first location, wherein sliding the collar distally adjusts said length and moves the flexible arm from the closed configuration to the open configuration.

5. The intrauterine device of claim 1, wherein the elongate member is a thread.

6. The intrauterine device of claim 1, wherein the elongate member is a semi-rigid rod.

7. The intrauterine device of claim 1, further comprising a stop member disposed between the first and second location when the flexible arm is in the closed configuration, wherein the stop member is disposed proximal to the first and second locations when the flexible arm is in the open configuration, and wherein the stop member is configured to maintain the flexible arm in the open configuration.

8. The intrauterine device of claim 7, wherein the elongate member is a thread, and the stop member is a knot.

9. The intrauterine device of claim 1, further comprising a contraceptive agent disposed on the elongate member between the first location and the second location.

10. An intrauterine device, comprising:
an elongate member; and
a flexible arm having a first end and a second end, wherein the first end is operatively coupled to the elongate member at a first location, and the second end is operatively coupled to the elongate member at a second location, wherein the flexible arm is adjustable between a closed configuration, in which the first location and the second location define a closed length, and an open configuration, in which the first location and the second location define an open length that is less than the closed length, and wherein the flexible arm is biased to be in the closed configuration.

11. The intrauterine device of claim 10, wherein the flexible arm is closely apposed to the elongate member when in the closed configuration, and wherein the flexible arm extends away from the elongate member when in the open configuration.

12. The intrauterine device of claim 10, further including a delivery apparatus connectable to the device, the delivery apparatus configured to engage the device in the closed configuration, and to move the device into the open configuration.

13. The intrauterine device of claim 10, wherein the elongate member comprises a contraceptive agent.

14. The intrauterine device of claim 12, wherein the delivery apparatus is steerable.

15. The intrauterine device of claim 14, wherein the delivery apparatus comprises pull wires.
